(19) United States
(12) Patent Application Publication
(10) Pub. No.: US 2009/0050824 A1
(43) Pub. Date: Feb. 26, 2009

(54) METHOD OF FABRICATING A STRUCTURE FROM DIAMOND MATERIAL OR DIAMOND-LIKE CARBON MATERIAL

(75) Inventors: Shane Huntington, Sydenham (AU); Steven Prawer, Caulfield (AU); Brant Gibson, Richmond (AU); Paolo Olivero, Carlton (AU); Andrew Greentree, Goburg (AU); Barbara Fairchild, Goburg (AU)

Correspondence Address: GANZ LAW, P.C., P O BOX 2200, HILLSBORO, OR, 97123, US

(73) Assignee: THE UNIVERSITY OF MELBOURNE, Parkville (AU)

(21) Appl. No.: 12/144,911

(22) Filed: Jun. 24, 2008

Related U.S. Application Data
(60) Provisional application No. 60/946,106, filed on Jun. 25, 2007.

(51) Int. Cl. A61N 5/00; G02B 6/00

(52) U.S. Cl. 250/492.3; 385/147

(57) ABSTRACT

A method of fabricating a structure from diamond material or diamond-like carbon material, and a structure according to the method, the method comprising the steps of imposing a structural transformation on the crystallographic structure of the material in a first region located at a first depth below a surface of the material; imposing a structural transformation on the crystallographic structure of the material in a second region located at a second depth below the surface of the material and above of the first region, the second depth being selected so that the first region is separated from the second region by a third region; and removing at least a portion of the material of the first and second regions; wherein the structural transformations are imposed so that the crystallographic structure of the third region is largely unaffected and the third region has opposite surface portions from which material of the first and second regions has been removed.

### RELATED APPLICATIONS

This application claims the benefit of and priority to U.S. Provisional Application Ser. No. 60/946,106, filed Jun. 25, 2007, the contents of which are hereby incorporated by reference as if recited in full herein for all purposes.

### BACKGROUND

The inventive subject matter broadly relates to a method of fabricating a structure from diamond material or diamond like carbon material. The inventive subject matter relates particularly, though not exclusively, to a method of fabricating a structure from single-crystalline diamond material.

Diamond material has properties that are ideally suited for a range of device applications such as those in the field of optics, photonics or electronics. In particular single crystalline diamond is very hard, is chemically inert, has a high optical refractive index and no grain boundaries.

In the past, methods have been proposed to carve structures into diamond material so that the advantageous properties of the diamond material can be exploited for some device applications. For example, the diamond material may be exposed to high energy ion irradiation, which locally damages the crystallographic structure in a region in which subsequently graphite is formed using suitable annealing procedures. The graphite may then be removed using etching techniques so that a structure is carved into the diamond material.

US patent application number 2006/0172515 discloses a method for fabricating layers that are undercut and only suspended at edge portions. The disclosed method can be used to fabricate such single crystalline diamond layers having a thickness in the order of a few μm. The diamond material is exposed to ion irradiation and the ion energy is selected so that the ions damage the crystallographic structure of the diamond at a depth of few μm below a surface layer. Graphite is then formed in the damaged region using annealing procedures and is subsequently removed so that the surface layer of the diamond material is undercut. Portions of the surface layer may then be removed and may be used for device fabrication and/or cantilever or bridge-structures may be formed in the diamond material.

However, for many device applications, in particular in the field of optics and photonics, thinner structures having a thickness in the sub-micron range are now required. Such devices, which may operate in the visible light range, may include for example photonic bandgap structures, single-mode waveguides and optical cavities. The fabrication of such very thin layers from diamond material in largely single crystalline form is a challenge and there is a need for technological advancement.

### SUMMARY

The inventive subject matter provides in a first aspect a method of fabricating a structure from diamond material or diamond-like carbon material, the method comprising the steps of:

imposing a structural transformation on the crystallographic structure of the material in a first region located at a first depth below a surface of the material;

imposing a structural transformation on the crystallographic structure of the material in a second region located at a second depth below the surface of the material and above the first region, the second depth being selected so that the first region is separated from the second region by a third region; and

removing at least a portion of the material of the first and second regions;

wherein the structural transformations are imposed so that the crystallographic structure of the third region is largely unaffected and the third region has opposite surface portions from which material of the first and second regions has been removed.

In one specific embodiment of the inventive subject matter the diamond material is substantially single-crystalline.

The structural transformation of the material in the first region typically is imposed before the structural transformation of the material in the second region.

The steps of imposing a structural transformation on the crystallographic structure of the material in the first and second regions typically comprise damaging the crystallographic structure using ion bombardment.

In a specific embodiment of the inventive subject matter the structural transformations of the first and second regions are imposed using respective ion bombardment conditions.

High energy ions, such as ions having an energy of one or more MeV, damage the crystallographic structure predominantly at a depth of one or more micrometers below the surface. Such high energy ions impose most of the damage within a rather narrow range. Most of the damage may be imposed within a region having a width of less than 200 nm or even less than 100 nm. For example, a beam of Helium ions having an energy of 2.0 MeV damages the crystallographic structure largely at a depth of approximately 3500 nm and a beam of Helium ions having an energy of 1.7 MeV damages the crystallographic structure largely at a depth of approximately 2900 nm. If the material is exposed to both ion bombardment treatments, an intermediate layer that is largely unaffected is located between the layers having the damaged crystallographic structure.

In one specific embodiment of the inventive subject matter the ion bombardment conditions for imposing the structural transformation in the first region include a first ion energy and the ion bombardment conditions for imposing the structural transformation in the second region include a second ion energy that is lower than the first ion energy. In this case the first and second ion energies are selected so that the structural transformations are effected in the first and second regions which are separated by the third region and the third regions is largely unaffected.

The method typically comprises selectively removing the first and second regions in a manner that will be described below. Further, any surface layer material that may be positioned over the first region typically is removed so that the third region is exposed.

By selecting conditions for imposing the structural transformations, such as ion beam energies and/or ion fluences, it is possible to control the thickness of the third region. As the regions within which the ions damage the crystallographic structure typically are highly confined to rather narrow regions, it is possible to select ion beam energies so that the third region is very thin.

For example, the conditions for imposing the crystallographic transformations may be selected so that the third region has a thickness of less than 1000 nm, less than 500 nm, less than 300 nm, less than 200 nm or less than 100 nm. The third region may then be removed from the diamond or diamond-like carbon material using a method that will be described below.

Consequently, the above-defined method has the significant practical advantage that (largely) single crystalline diamond material can be fabricated having a thickness in the sub-micron range. This enables fabrication of devices that require such thin materials and would benefit from the superior properties of (largely) single crystalline diamond.

In a specific embodiment of the inventive subject matter the method comprises the step of controlling the thickness of the third region by controlling the respective conditions which are used to impose the structural transformation of the material in the first and second regions. Typically, this comprises controlling respective energies of the ion bombardments which typically are used to impose the structural transformations.

Further, the method may comprise the additional step of controlling the thickness of the third region by controlling the temperature of the diamond or diamond-like carbon material during the ion bombardment treatments.

The method may also include applying a mask to the diamond or diamond-like carbon material prior to at least one of the ion bombardment treatments so as to control an extension of a region within which the at least one ion bombardment treatment is imposed.

The method may also include the additional step of annealing the material after imposing the structural transformations. The annealing conditions and the conditions for the structural transformations typically are selected so that graphite is formed in the first and second regions.

The method may also comprise the step of forming a conduit for a fluid to the first and second regions. For example the conduit may be formed using a focussed ion, electron or laser beam.

Further, the second region typically is positioned below a surface layer in which structural transformation is reduced or does not occur.

The step of removing the first and second regions typically comprises etching, such as chemical etching, electro-chemical etching, plasma etching or exposing the sample to hot gases such as hot oxygen. In this case an etch fluid, such as an etch liquid, may be directed through the conduit to the first and second regions and selected so that material of the first and second regions is removed by etching.

The surface layer may be removed using suitable cutting techniques that may employ focussed ion, laser or electron beams after removing the first and second regions.

In a specific example the graphite is removed using a wet-chemical etch process that selectively etches graphite and has a significantly lower etch rate for diamond.

The method may comprise a further annealing step after the material of the first and second regions has been removed. This annealing step may be conducted at a relatively high temperature, such as a temperature of more than 1000° C., which reduces damage that the ion bombardment may have caused in the third region.

The method typically comprises removing at least a portion of the third region by cutting the third region. The removed portion of the third region may then be used for device fabrication. For example the cutting may comprise forming a profiled structure from material of the third region, such as a ring cavity or any other structure, by directing an ion, electron or laser beam along an appropriate path.

Alternatively or additionally, at least a portion of the third region may remain attached to the remaining diamond or diamond-like carbon material. In this case for example a bridge or cantilever structure may be formed from the material of the third region. The cutting may comprise using a focussed ion, electron or laser beam having sufficient energy.

It is to be appreciated that in variations of the above-described embodiments structural transformations may also be imposed within more than two regions which are separated by regions in which structural transformations are largely avoided. In this way a stack of thin largely single crystalline regions may be formed. For example, ion bombardment treatments may be performed with three or more ion energies, which are selected so that regions in which crystallographic damage is imposed are separated by regions which are largely free from structural damage.

The inventive subject matter provides in a second aspect a structure fabricated by the method according to the first aspect of the inventive subject matter.

The structure may be a high frequency resonator. As diamond is a very hard material, the resonator has the advantage of having a high resonance frequency if sufficiently small proportioned.

The structure may also be an optical device. For example, the optical device may comprise any type of waveguide including cavities such as ring cavities, or a photonic crystal structure.

In one specific embodiment the optical device comprises a body portion and a waveguide overhanging a region of the body portion, wherein the body portion and the waveguide are formed from single crystalline diamond.

The waveguide may also comprise a photon source such as any type of colour centre including those having at least one optically active impurity atom.

The inventive subject matter will be more fully understood from the following description of specific embodiments of the inventive subject matter. The description is provided with reference to the accompanying drawings.

### BRIEF DESCRIPTION OF THE DRAWINGS

FIG. 1 shows calculated structural damage imposed by Helium ions as a function of depth for diamond material;

FIG. 2 shows a structure formed using a method according to an embodiment of the inventive subject matter; and

FIG. 3 (a)-(c) shows structures that were fabricated using a method according to a specific embodiment of the inventive subject matter.

### DETAILED DESCRIPTION OF SPECIFIC EMBODIMENTS

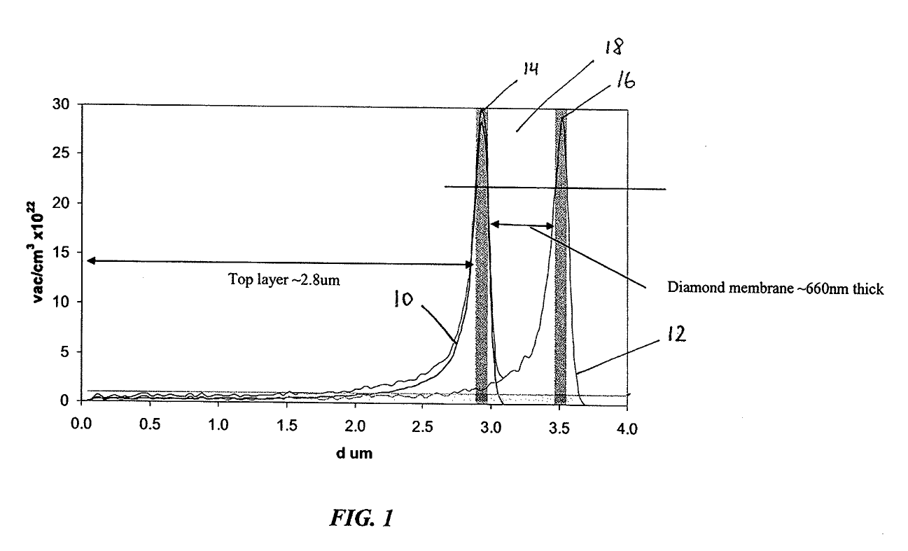

Referring initially to FIG. 1, a method of fabricating a structure from diamond material or diamond like carbon material according to a specific embodiment of the inventive subject matter is now described.

The method according to the specific embodiment uses ion bombardment of the material to damage the crystallographic structures at selected depths below a surface of the material. FIG. 1 shows plots 10 and 12 indicating the damage that has been caused by the ion bombardment as a function of depth below the surface of single crystalline diamond material.

The plots 10 and 12 show data which were obtained using a Stopping and Range of Ions in Matter (SRIM) simulation for ion beam energies of 1.7 MeV (plot 10) and 2.0 MeV (plot 12). The plots 10 and 12 show vacancies per cm³ that are generated by the ions as a function of depth. As can be seen from plots 10 and 12, the damaged regions are rather narrow and have a thickness of the order of 100 nm. Helium ions having an energy of 2.0 MeV damage the crystallographic structure mainly at a first region 16 located at a depth of 3500 nm and Helium ions having an energy of 1.7 MeV damage the crystallographic structure mainly at second region 14 located at a depth of approximately 2900 nm. A third region 18 is located between the second region 14 and the first region 16 and the third region 18 is largely free from damage imposed by ion bombardment. The third region 18 has a thickness of approximately 660 nm. As can be seen from the plots 10 and 12, a top layer having a thickness of approximately 2800 nm is also largely undamaged.

It is known that above a threshold vacancy density diamond is at least partially converted into graphite if subsequently annealed. The ion bombardment conditions are selected so that the material of regions 14 and 16 will convert to graphite. However, region 18 and the top layer will remain largely unaffected.

It will be appreciated that in variations of this embodiment other ion energies may be chosen so as to control the thickness of the region 18. For example, the ion energies may be controlled so that the region 18 has a thickness of less then 300 nm or less than 200 nm or less than 100 nm.

Further, the inventors have observed that the threshold vacancy density is dependent on the temperature of the diamond material during the ion irradiation. If the temperature is increased, the threshold vacancy density typically also increases. Consequently, the damage that is being imposed by the ion bombardment is confined to an even narrower region if the temperature of the diamond material is increased and it is possible to control the thickness of the regions 14 and 16, and consequently the thickness of the region 18, by controlling the temperature of the diamond material during the irradiation. For example, for imposing the damage on the crystallographic structure of the first region a first temperature may be selected and for imposing the damage on the crystallographic structure of the second region a second temperature may be selected. This allows controlling the thicknesses of the first and second regions independent from one another.

In a specific embodiment of the inventive subject matter single crystalline diamond is treated by ion bombardment in the above-described manner. The material is then annealed at a temperature of 550° C. for one hour in air, which results in formation of graphite in regions 14 and 16.

A conduit is then formed to the regions 14 and 16 so that an etching fluid can be transported to the regions 14 and 16. For example, the conduit may comprise one or more bores. Alternatively, any other type of structure may be written into the diamond material and may function as conduit to the regions 14 and 16. In this embodiment, the conduit is formed by exposing the diamond material to a focussed beam of 30 keV Gallium ions having a beam current of approximately 1 to 2 nA, a beam size of approximately 1000 nm, and a milling rate of 100 nm³/nC. Alternatively, a suitable electron beam or laser beam may be used.

The material is then exposed to a boiling acid solution comprising one part H₂SO₄, one part HNO₃ and one part HClO₄. This solution is known to preferentially etch graphite. Once the regions 14 and 16 are etched away, the top layer and region 18 were undercut.

The above-described process undercuts the region 18 and forms a single-crystalline diamond region that is suspended only at edges and has a thickness in the sub-micron range.

Initially sections are cut through the top layer so that the top surface of the region 18 can be imaged using electron microscopy and the remaining cutting process can be controlled using real time imaging. Once approximately 80% of the perimeter of a desired portion in the top layer is cut, the desired portion of the top layer is mechanically lifted off using a micro-tip welded to the top layer.

The diamond material is annealed in forming gas (4% hydrogen in argon) at a temperature of approximately 1100° C. for approximately two hours. This annealing process heals defects that may have been formed in the thin diamond region 18 when the material was exposed to bombardment by the high energy ions.

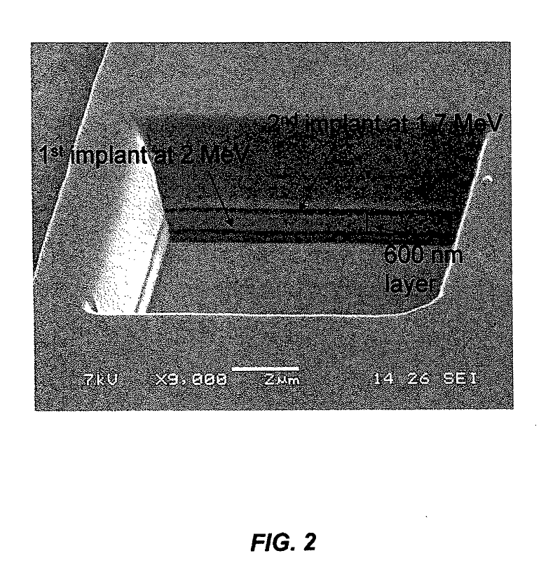

Structures may then be written into the region 18 and the region 18 may partially or entirely be removed and used for device fabrication. FIG. 2 shows the diamond material having a crater from which the top layer, regions 14, 18 and 16 were removed. In this case ion beams having energies of 1.7 MeV and 2 MeV were used to impose the structural transformations and the removed diamond material region 18 had a thickness of approximately 600 nm.

In the above-described example the region 18 is suspended at edges and positioned in the diamond material. In a variation of the described embodiment the conduit may also comprise a cut around the top layer and around the regions 14, 16 and 18. The etching process would then remove the material of the first and second regions and the region 18 would be unattached to the remaining diamond material.

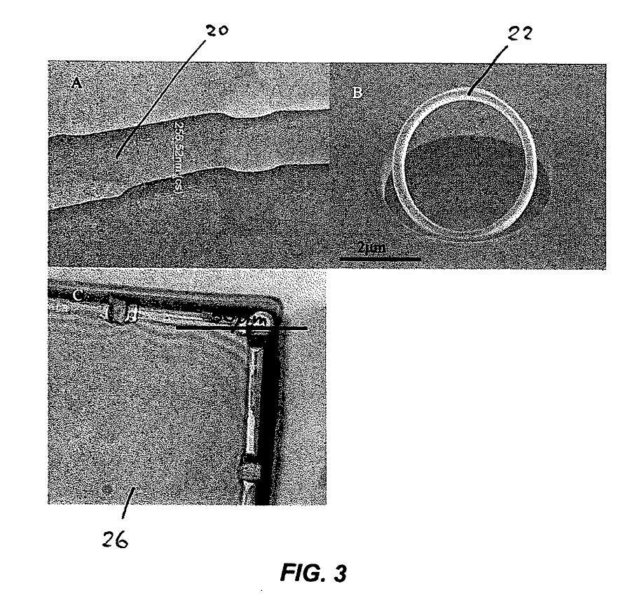

FIG. 3 (a) shows a cross-sectional representation of a layer 20 of single crystalline diamond that has a thickness of approximately 250 nm and was prepared in the above-described manner using Helium ion beam energies of 1.85 MeV and 2.0 MeV. For example, an optical waveguide may be formed from layer 20 which may comprise a photon source, such as a colour centre.

FIG. 3 (b) shows a ring cavity 22 that was formed by writing a ring-structure into a single crystalline layer of diamond material prepared in the above-described manner. The ring cavity has an outer diameter of 3600 nm, a ring width of 300 nm and depth of 330 nm.

FIG. 3 (c) shows Newton rings in a single crystalline diamond layer 26 having a thickness of 330 nm prepared in the above-defined manner.

Although the inventive subject matter has been described with reference to particular examples, it will be appreciated by those skilled in the art that the inventive subject matter may be embodied in many other forms. For example, a stack of thin diamond layers may be formed in a similar manner. This may comprise using a sequence of differing ion energies for the ion bombardment treatments, which would result in a stack of layered regions having a damaged crystallographic structure and which can be removed in the above-described manner. Further, the material may not necessarily be single crystalline diamond material, but may be a polycrystalline material such as a diamond-like carbon material.

Further, the top-surface may be masked prior to ion bombardment treatment of the diamond or diamond-like carbon material so that only a selected region is ion bombardment treated and the third region is formed with a shape that corresponds to the unmasked region. In addition, masking and ion bombardment processing steps may also be used to write structures into the third region by selectively imposing structural transformations within the third regions. This way a patterned third regions can be formed.

The reference that is being made to US patent application number 2006/0172515 does not constitute an admission that US patent application number 2006/0172515 is part of the common general knowledge in the US or in any other country.

All patent and non-patent literature cited herein is hereby incorporated by references in its entirety for all purposes.

1. A method of fabricating a structure from diamond material or diamond-like carbon material, the method comprising the steps of:
imposing a structural transformation on the crystallographic structure of the material in a first region located at a first depth below a surface of the material;
imposing a structural transformation on the crystallographic structure of the material in a second region located at a second depth below the surface of the material and above of the first region, the second depth being selected so that the first region is separated from the second region by a third region; and
removing at least a portion of the material of the first and second regions;
wherein the structural transformations are imposed so that the crystallographic structure of the third region is largely unaffected and the third region has opposite surface portions from which material of the first and second regions has been removed.

2. The method of claim 1 wherein the material is a substantially single-crystalline diamond material.

3. The method of claim 1 wherein the structural transformation of the material in the first region is imposed before the structural transformation of the material in the second region.

4. The method of claim 1 wherein the steps of imposing a structural transformation on the crystallographic structure of the material in the first and second regions comprises damaging the crystallographic structure using ion bombardment.

5. The method of claim 4 wherein the structural transformations of the first and second regions are imposed using respective ion bombardment conditions.

6. The method of claim 5 wherein the respective ion bombardment conditions for imposing the structural transformation in the first region include a first ion energy and the ion bombardment conditions for imposing the structural transformation in the second region include a second ion energy that is lower than the first ion energy, and wherein the first and second ion energies are selected so that the structural transformations are effected in the first and second regions which are separated by the third region and the third regions is largely unaffected.

7. The method of claim 5 wherein the conditions for imposing the crystallographic transformations are selected so that the third region has a thickness of less than 1000 nm.

8. The method of claim 5 wherein the conditions for imposing the crystallographic transformations are selected so that the third region has a thickness of less than 500 nm.

9. The method of claim 5 wherein the conditions for imposing the crystallographic transformations are selected so that the third region has a thickness of less than 300 nm.

10. The method of claim 5 wherein the conditions for imposing the crystallographic transformations are selected so that the third region has a thickness of less than 200 nm.

11. The method of claim 5 wherein the conditions for imposing the crystallographic transformations are selected so that the third region has a thickness of less than 100 nm.

12. The method of claim 1 comprising the step of controlling the thickness of the third region by controlling conditions which are used to impose the structural transformations of in the first and second regions.

13. The method of claim 12 wherein the structural transformations of the first and second regions are imposed by ion bombardment treatments and wherein the conditions include ion energies.

14. The method of claim 12 wherein the structural transformations of the first and second regions are imposed by ion bombardment treatments and wherein the conditions include ion fluences.

15. The method of claim 12 wherein the structural transformation of the first and second regions are imposed by ion bombardment treatments and wherein the conditions include the temperature of the diamond or diamond-like carbon material during the ion irradiation.

16. The method of claim 1 comprising the step of forming a conduit for a fluid to the first and second regions.

17. The method of claim 1 comprising annealing the material after the structural transformations have been imposed, wherein the conditions for imposing the structural transformations and conditions for the annealing are selected so that graphite is formed in the first and the second regions.

18. The method of claim 1 wherein the step of removing the first and second regions comprises etching.

19. The method of claim 17 wherein the step of removing the first and second regions comprises etching and wherein the etching is selected so that predominantly the graphite is etched.

20. The method of claim 18 comprising the step of forming a conduit for a fluid to the first and second regions and wherein the etching comprises directing an etch fluid through the conduit to the first and second regions.

21. The method of claim 1 comprising removing material above the first region.

22. The method of claim 1 comprising removing at least a portion of the third region by cutting the third region.

23. The method of claim 22 comprising using the removed portion of the third region for device fabrication.

24. The method of claim 23 comprising using at least a portion of the third region that remains attached to the remaining diamond or diamond-like carbon material for device fabrication.

25. The method of claim 1 wherein the structural transformations are imposed using ion bombardment treatments and comprising applying a mask to the diamond or diamond-like carbon material prior to at least one of the ion bombardment treatments so as to control an extension of a region within which the at least one ion bombardment treatment is imposed.

26. A structure fabricated by the method according to claim 1.

27. The structure of claim 26 wherein the structure is a high frequency resonator.

28. The structure of claim 26 wherein the structure is an optical device.

29. The structure of claim 26 wherein the optical device is a waveguide.

30. The structure of claim 26 wherein the optical device is a cavity.

31. The structure of claim 26 wherein the optical device is a photonic crystal structure.

32. The structure of claim 26 wherein the waveguide comprises a photon source.

33. The structure of claim 32 wherein the photon source comprises a colour centre.
